(19) United States
(12) Patent Application Publication
(10) Pub. No.: US 2026/0021218 A1
(43) Pub. Date: Jan. 22, 2026

(54) OPTOELECTRONIC DEVICE

(71) Applicant: ams-OSRAM International GmbH, Regensburg (DE)

(72) Inventors: Alexander WILM, Regensburg (DE); Peter WOLFF, Donaustauf (DE)

(21) Appl. No.: 18/993,477

(22) PCT Filed: Jul. 5, 2023
(86) PCT No.: PCT/EP2023/068486

§ 371 Date: Jan. 10, 2025

(30) Foreign Application Priority Data
Jul. 22, 2022 (DE) 10 2022 118 392.0

(51) Int. Cl. A61L 9/20; A61L 2/10; F21V 8/00

(52) U.S. Cl. CPC A61L 9/20; A61L 2/10; G02B 6/0043; G02B 6/0068; A61L 2202/11; A61L 2209/12

(57) ABSTRACT

An optoelectronic device is specified herein, comprising at least one emitter configured for emitting electromagnetic radiation in an ultraviolet spectral range, and at least one optical element configured for redirecting the electromagnetic radiation onto an external surface, such that the external surface is uniformly irradiated.

An optoelectronic device is specified herein.

At least one object of certain embodiments is to specify an optoelectronic device for a uniform irradiation of an external surface with electromagnetic radiation.

According to at least one embodiment the optoelectronic device comprises at least one emitter configured for emitting electromagnetic radiation in an ultraviolet spectral range. For example, the emitter emits electromagnetic radiation with a wavelength in a range between 100 nm and 400 nm, inclusive. Preferably, the emitter emits electromagnetic radiation in a UV-C spectral range with a wavelength in a range between 100 nm and 280 nm, inclusive. In particular, the emitter is configured to convert an electric current into the electromagnetic radiation during operation.

According to at least one embodiment, the optoelectronic device comprises at least one optical element configured for redirecting the electromagnetic radiation onto an external surface, such that the external surface is uniformly irradiated. For example, the optical element redirects the electromagnetic radiation due to refraction, reflection, diffraction, scattering, and/or interference of the electromagnetic radiation.

Here and in the following the external surface is irradiated “uniformly”, if an intensity of the electromagnetic radiation changes by at most 40%, preferably by at most 20%, or particularly preferably by at most 10% across the irradiated external surface. In other words, a difference between a maximal intensity and a minimal intensity of the electromagnetic radiation in different regions of the irradiated external surface is smaller than or equal to 40%, preferably smaller than or equal to 20%, or particularly preferably smaller than or equal to 10% of an average intensity of the electromagnetic radiation across the irradiated surface.

According to a preferred embodiment, the optoelectronic device comprises:
at least one emitter, configured for emitting electromagnetic radiation in an ultraviolet spectral range, and
at least one optical element configured for redirecting the electromagnetic radiation onto an external surface, such that the external surface is uniformly irradiated.

The optoelectronic device described herein advantageously provides a uniform surface irradiation while giving rise to a slim form factor. For example, a distance between the optoelectronic device and the external surface is smaller than a length and/or a width of the external surface that is uniformly irradiated by the electromagnetic radiation. Moreover, the distance between the optoelectronic device and the external surface may be smaller than a distance between two emitters of the optoelectronic device. Further, due to the optical element a smaller number of emitters may be needed for a uniform surface irradiation compared to an optoelectronic device without an optical element, thereby reducing a cost of the optoelectronic device.

A uniform surface irradiation is advantageous for UV disinfection applications. In optoelectronic devices for UV disinfection applications, where emitters directly irradiate the external surface and no optical element for redirecting the electromagnetic radiation is arranged in between, a lateral distance between different emitters is approximately equal to a vertical distance between an emitter and the irradiated surface in order to achieve a uniform surface irradiation. Here “lateral” refers to a direction parallel to the external surface, whereas “vertical” refers to a direction perpendicular to the external surface.

If the available space for arranging the optoelectronic device is constrained in the vertical direction, for example in compact air conditioning units, a large number of emitters may be needed to achieve uniform surface irradiation together with a small vertical distance between the emitters and the external surface. In this case a necessary irradiation intensity for disinfection applications may be significantly surpassed, and/or the optoelectronic device may have a high cost due to a large number of emitters needed. Advantageously, the optoelectronic device described herein allows to reduce the number of emitters, while keeping the vertical distance between the emitters and the external surface as small as possible.

According to at least one embodiment of the optoelectronic device, the emitter is a light-emitting semiconductor diode. In particular, the light-emitting semiconductor diode comprises a semiconductor layer stack with a pn-junction for converting an electric current into electromagnetic radiation. For example, the semiconductor layer stack comprises a III/V compound semiconductor material.

A III/V compound semiconductor material comprises at least one element from the third main group, such as B, Al, Ga, In, and one element from the fifth main group, for example N, P, As. In particular, the term “III/V compound semiconductor material” includes the group of binary, ternary or quaternary compounds containing at least one element from the third main group and at least one element from the fifth main group. Moreover, the III/V semiconductor material may comprise one or more dopants.

Preferably, the semiconductor layer sequence comprises or consists of a nitride compound semiconductor material. Nitride compound semiconductor materials are III/V compound semiconductor materials comprising nitrogen, such as materials from the system InₓAl_{y}Ga_{1-x-y}N with 0≤x≤1, 0≤y≤1 and x+y≤1.

According to at least one embodiment, the optoelectronic device comprises at least two emitters, wherein a distance between the two emitters is at least two times larger, preferably at least five times larger, than a minimal distance between one of the emitters and the external surface. In particular, the distance between the two emitters is a lateral distance, i.e. a distance in a direction parallel to the external surface. Moreover, the minimal distance between one of the emitters and the external surface is preferably a vertical distance, i.e. a distance in a direction perpendicular to the external surface.

According to at least one embodiment of the optoelectronic device, the optical element comprises a light guide with a light incoupling surface and a light outcoupling surface for the electromagnetic radiation, and the light outcoupling surface is larger than the light incoupling surface by at least a factor of two, preferably by at least a factor of ten. The light incoupling surface and/or the light outcoupling surface may be planar or curved. In particular, the electromagnetic radiation is coupled into the light guide via the light incoupling surface and the electromagnetic radiation is coupled out of the light guide via the light outcoupling surface during operation of the optoelectronic device. In particular, an area of the light outcoupling surface is larger than an area of the light incoupling surface by at least a factor of two, preferably by at least a factor of ten.

In particular, the light guide comprises or consists of a material that is at least partially transparent for the electromagnetic radiation emitted by the emitter. For example, the light guide comprises quartz glass.

The light guide is quasi one-dimensional or quasi two-dimensional, for example. Here and the following “quasi one-dimensional” refers to a light guide with a spatial extension in one direction much larger than a spatial extension in two complementary perpendicular directions. For example, the spatial extension in one direction is at least ten times larger than the spatial extension in the other two directions. Here and in the following “quasi two-dimensional” refers to a light guide with the spatial extension in one direction much smaller than the spatial extension in two complementary perpendicular directions. For example, the spatial extension in one direction is at most a tenth of the spatial extension in the other two directions.

For example, the light guide has the form of a cylinder or of a thin sheet. A cross-section of the cylinder may be circular, semi-circular, elliptical, rectangular or square, for example. Here, the cross-section preferably refers to a shape of a base surface of the cylinder. In particular, the light incoupling surface corresponds to one or both of the flat base surfaces on opposite ends of the cylinder, whereas the light outcoupling surface corresponds to a lateral outer surface of the cylinder. Preferably, the lateral outer surface connects the two base surfaces on opposite ends of the cylinder.

According to at least one embodiment of the optoelectronic device, the light outcoupling surface has a structuring such that the electromagnetic radiation is scattered out of the light guide. In particular, the electromagnetic radiation is diffusively scattered out of the light guide. For example, the electromagnetic radiation propagates inside the light guide parallel to the light outcoupling surface. In order to extract the electromagnetic radiation out of the light guide, the light outcoupling surface comprises a structuring in the form of a surface roughening, for example. In particular, the light outcoupling surface comprises a plurality of recesses.

The features of a single recess described in the following apply to a majority of recesses, preferably to all recesses. Preferably, a depth of the recess in a direction perpendicular to the light outcoupling surface is larger than the wavelength of the electromagnetic radiation, such that the electromagnetic radiation is diffusively scattered at the recess. Different recesses may have different shapes or may have the same shape within manufacturing tolerances. The shape of the recess may be random or regular, such as cylindrical, cuboid or pyramidal, for example. In particular, the recesses are randomly or regularly distributed across the light outcoupling surface. A surface of the light guide opposite to the light outcoupling surface may also comprise a structuring as described above.

According to at least one embodiment of the optoelectronic device, the structuring is configured to compensate an intensity gradient of the electromagnetic radiation inside the light guide, such that an intensity of the outcoupled electromagnetic radiation is uniform across the light outcoupling surface. In particular, the intensity of the electromagnetic radiation may decrease along the light guide with increasing distance from the light incoupling surface, giving rise to the intensity gradient inside the light guide.

For example, an area density of recesses in the light outcoupling surface increases with an increasing distance from the light incoupling surface, such that the scattering of the electromagnetic radiation out of the light guide is stronger at larger distances from the light incoupling surface. Moreover, a distance between the light outcoupling surface and the external surface may decrease with increasing distance from the light incoupling surface, in order to compensate the intensity gradient inside the light guide.

According to at least one embodiment of the optoelectronic device, the optical element comprises light scattering particles embedded in a transparent matrix material. In particular, the light scattering particles change the propagation direction of at least a part of the electromagnetic radiation propagating inside the optical element. Preferably, an average size of the light scattering particles is smaller than or equal to the wavelength of the electromagnetic radiation.

Preferably, the light scattering particles are distributed along the light guide such that an intensity of the outcoupled electromagnetic radiation is uniform across the light outcoupling surface. For example, a number of the light scattering particles per volume increases with increasing distance from the light incoupling surface.

The matrix material may comprise a glass, for example quartz glass. In particular, the transparent matrix material is at least partially transparent for the electromagnetic radiation generated by the emitter during operation. For example, the matrix material absorbs at most 10% of the electromagnetic radiation coupled into the light guide after an optical path length of approximately 10 mm.

The matrix material may also comprise a plurality of air-bubbles. In particular, an air-bubble is a closed cavity inside the matrix material which is preferably filled with air. For example, the air-bubbles change the propagation direction of at least a part of the electromagnetic radiation propagating inside the optical element. Preferably, an average size of the air-bubbles is smaller than or equal to the wavelength of the electromagnetic radiation. In particular, the air-bubbles may scatter the electromagnetic radiation similar or equal to the scattering particles.

According to at least one embodiment of the optoelectronic device, the optical element comprises a reflective element. For example, the reflective element comprises or consists of a reflective surface coating and/or a mirror. The reflective element may be planar or curved. For example, the reflective element comprises a surface coating on a part of the light outcoupling surface of the light guide. In particular, the reflective surface coating comprises a metal, such as Aluminium, for example.

For example, the reflective surface has no focal point, one focal point, or at least two focal points. The reflective surface may also have a plurality of focal points. In particular, the reflective surface may comprise a plurality of regions or sections, where each region or section has a separate focal point. For example, the plurality of focal points are arranged densely along a line, such that they form a focal line. The focal line may be a straight line or a curved line. The curved focal line may be a curved line within a two-dimensional plane, or may be a curved line within a three-dimensional space. The plurality of focal points may also be arranged within a two-dimensional plane without forming a focal line. For example, the emitter is arranged at least at one focal point, or the emitter is arranged off-centered from at least one focal point, or the emitter is arranged off-centered from all focal points of the reflective surface.

According to at least one embodiment of the optoelectronic device, the emitter and the reflective element do not overlap with the external surface in a plan view of the external surface and/or in a side view of the external surface. In particular, here and in the following “plan view” refers to a view along a direction perpendicular to the external surface, whereas “side view” refers to a view along a direction parallel to the external surface.

For example, a fluid flows through the external surface or flows parallel to the external surface and the electromagnetic radiation emitted by the optoelectronic device is configured for disinfecting the fluid. By arranging the optoelectronic device such that it does not overlap with the external surface, the fluid may flow without being obstructed by the optoelectronic device.

According to at least one embodiment of the optoelectronic device, an area of the external surface is at least ten times larger, preferably at least twenty times larger, than an area of a reflective surface of the reflective element. Advantageously, the optoelectronic device thus has a compact size compared to the external surface.

According to at least one embodiment of the optoelectronic device, the reflective surface is a freeform surface. In particular, a shape of the reflective surface is configured such that the electromagnetic radiation emitted by the emitter is redirected onto the external surface and that the external surface is uniformly irradiated. Preferably, the freeform surface is curved. For example, the freeform surface has different curvatures along different directions. In particular, the freeform surface may be neither hyperbolically, nor parabolically, nor spherically shaped.

According to at least one embodiment of the optoelectronic device, the external surface has an aspect ratio larger than 2, preferably larger than 5 and the reflective element collimates the electromagnetic radiation along a short axis of the external surface and homogenizes an intensity profile of the electromagnetic radiation along a long axis of the external surface. Here and in the following “aspect ratio” refers to a ratio between a maximal diameter and a minimal diameter of the external surface. Moreover, “long axis” refers to a direction in which the diameter is maximal, whereas “short axis” refers to a direction in which the diameter is minimal.

In particular, the collimated electromagnetic radiation propagates approximately parallel with a small beam divergence across the short axis. For example, the beam divergence is at most 30°, preferably at most 20°, or particularly preferably at most 10°. In other words, the electromagnetic radiation propagates inside an angular cone with on opening angle of at most 10° across the short axis. Moreover, the homogeneous intensity profile along the long axis gives rise to the uniform surface irradiation.

Further a fluid cooling system is specified. In particular, the fluid cooling system comprises an optoelectronic device as described above. All features of the optoelectronic device are also disclosed for the fluid cooling system and vice versa.

According to at least one embodiment, the fluid cooling system comprises an optoelectronic device as described above. In particular, the optoelectronic device is configured to prevent the formation of a film of biological material on parts of the fluid cooling system due to irradiation with ultraviolet electromagnetic radiation. Moreover, the optoelectronic device may at least partially disinfect a fluid flowing through the fluid cooling system during operation. For example, the electromagnetic radiation emitted by the optoelectronic device during operation inactivates or destroys at least 70%, preferably at least 90%, or particularly preferably at least 99% of bacteria and/or viruses in the fluid flowing through the fluid cooling system.

According to at least one embodiment, the fluid cooling system comprises at least two cooling fins arranged parallel to each other, configured to cool a fluid flowing between the two cooling fins, wherein the electromagnetic radiation emitted by the optoelectronic device irradiates the fluid between the two cooling fins during operation. In particular, the two cooling fins have a lower temperature than the fluid, for example. Preferably, the fluid may be air flowing between the two cooling fins. The fluid may also be a liquid flowing between the cooling fins. For example, the fluid cooling system is an air conditioning system.

According to at least one embodiment of the fluid cooling system, the optoelectronic device is arranged outside a volume delimited by the two cooling fins. For example, the two cooling fins have the same shape. In this case the two cooling fins partially enclose a volume that is given by an area of a cooling fin times a distance between the two cooling fins, for example.

The fluid cooling system may also comprise a plurality of cooling fins that are arranged parallel to each other. In this case, the optoelectronic device may be arranged outside a volume spanned by the plurality of cooling fins. Advantageously, by arranging the optoelectronic device outside the volume spanned by the plurality of cooling fins, the fluid flow between the cooling fins is not obstructed by the optoelectronic device.

According to at least one embodiment of the fluid cooling system the external surface irradiated by the electromagnetic radiation during operation corresponds to a cross-sectional surface of the volume through which the fluid flows. In particular, the cross-sectional surface is arranged perpendicular to a main extension plane of the cooling fins.

According to at least one embodiment of the fluid cooling system, the optoelectronic device is arranged between the two cooling fins such that the external surface irradiated by the electromagnetic radiation during operation corresponds to a main surface of at least one of the cooling fins.

Further advantageous embodiments and further embodiments of the optoelectronic device and the fluid cooling system will become apparent from the following exemplary embodiments described in connection with the figures.

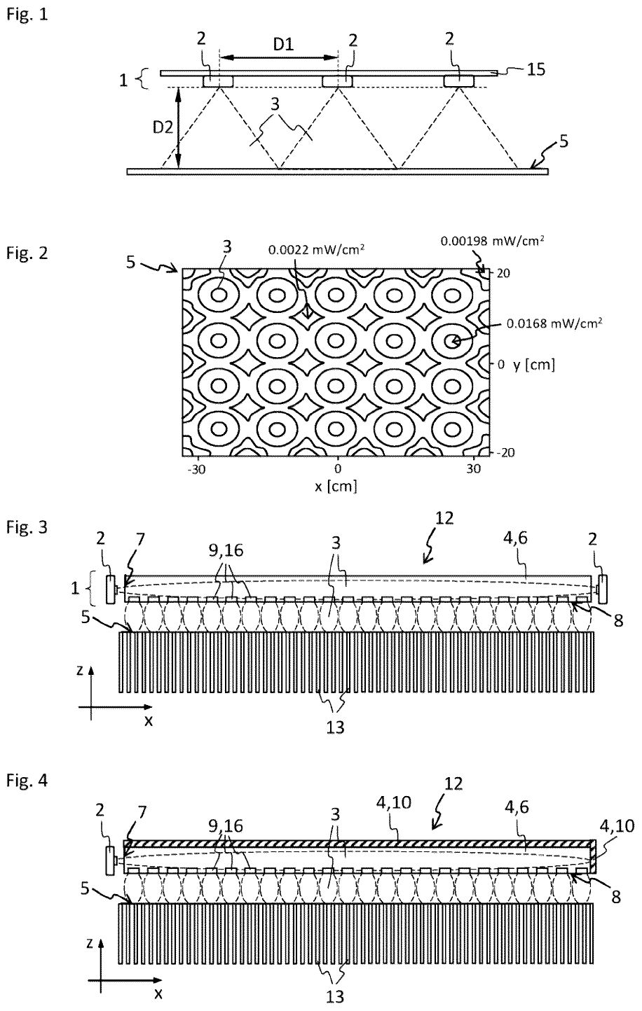

FIG. 1 shows a schematic cross-section of an optoelectronic device according to an example.

FIG. 2 shows a schematic intensity distribution of electromagnetic radiation across an external surface according to an example.

FIGS. 3 to 7 show schematic cross-sections of fluid cooling systems according to different exemplary embodiments.

FIGS. 8 to 17 show different schematic cross-sections of fluid cooling systems according to further exemplary embodiments.

Elements that are identical, similar or have the same effect are denoted by the same reference signs in the figures. The figures and the proportions of the elements shown in the figures are not to be regarded as true to scale. Rather, individual elements may be shown exaggeratedly large for better representability and/or better understanding.

The optoelectronic device 1 according to the example in FIG. 1 comprises a plurality of emitters 2 arranged on a main surface of a carrier 15. The emitters 2 are light-emitting diodes that emit electromagnetic radiation 3 preferably in a direction perpendicular to the main surface of the carrier 15. In particular, the emitters 2 are configured to uniformly irradiate an external surface 5 that is arranged parallel to the main surface of the carrier 15 at a distance D2 from the emitters 2.

In order to uniformly irradiate the external surface 5, a lateral distance D1 between neighboring emitters 2 is approximately equal to the distance D2 between the emitter 2 and the external surface 5. If the external surface 5 has a given area that needs to be uniformly irradiated by the electromagnetic radiation 3, the distance D2 between the emitter 2 and the external surface 5 thus depends on the number of emitters 2 in the optoelectronic device 1. In particular, for a smaller number of emitters 2, the distance D1 between the emitters 2 and thus the distance D2 between the emitter 2 and the external surface 5 needs to increase in order to achieve a uniform irradiation of the external surface 5.

If a maximum distance D2 between the emitter 2 and the external surface 5 is limited by mechanical constraints, a large number of emitters 2 might be necessary for a uniform irradiation of the external surface 5, thereby increasing the cost of the optoelectronic device 1. Moreover, the intensity of the electromagnetic radiation 3 on the external surface 5 may surpass a necessary irradiation intensity needed for disinfection.

A schematic irradiance distribution of electromagnetic radiation 3 across an irradiated external surface 5 is shown as a contour plot as function of x and y coordinates of the external surface 5 in FIG. 2. In particular, the irradiance distribution corresponds to the setup shown in FIG. 1, if the distance D2 between the emitter 2 (not shown) and the external surface 5 is smaller than the distance D1 between neighbouring emitters 2. Accordingly, the irradiance distribution across the external surface 5 is not uniform. In particular, the irradiance of the electromagnetic radiation 3 at a position of maximal irradiance is approximately by a factor of ten larger, than at a position on the external surface 5 where the irradiance of electromagnetic radiation is lowest. In particular, disinfection might not be effective at positions on the external surface 5 where the irradiance of the electromagnetic radiation 3 is too low.

The fluid cooling system 12 according to the exemplary embodiment of FIG. 3 comprises an optoelectronic device 1 and a plurality of cooling fins 13. The optoelectronic device 1 is arranged outside a volume spanned by the plurality of cooling fins 13.

Here and in the following figures a Cartesian coordinate system with x, y and z coordinates that are perpendicular to each other is used to describe the orientation of various elements. In particular, FIG. 3 shows a schematic cross-section of the fluid cooling system 12 in the x-z plane. The cooling fins 13 have main extension planes parallel to the y-z plane, whereas the external surface 5 is parallel to the x-y plane and corresponds to a side surface of the volume spanned by the plurality of cooling fins 13.

Electromagnetic radiation 3 is emitted by the optoelectronic device 1 preferably against the z-direction, such that the electromagnetic radiation 3 can propagate between the cooling fins 13. The optoelectronic device 1 comprises an optical element 4 in the form of a light guide 6 arranged between two emitters 2 that are light-emitting semiconductor diodes. The two emitters 2 emit electromagnetic radiation 3 in a UV-C spectral range during operation. The electromagnetic radiation 3 is coupled into the light guide 6 via two light incoupling surfaces 7 on opposite ends of the light guide 6. The light incoupling surfaces 7 are arranged parallel to the y-z plane.

The light guide 6 has a quasi one-dimensional cylindrical form extending in the x direction or is quasi two-dimensional in the form of a thin sheet extending in x and y directions. In particular, a spatial extension of the light guide 6 between the two emitters 2 in x-direction is at least larger by a factor of 10 than a thickness of the light guide 6 in z-direction.

The light outcoupling surface 8 of the light guide 6 extends in x-direction and is arranged parallel to the propagation direction of electromagnetic radiation 3 inside the light guide 6. In order to couple the electromagnetic radiation 3 out of the light guide 6 via the light outcoupling surface 8, the light outcoupling surface 8 comprises a structuring 9. In particular, the structuring 9 comprises a plurality of recesses 16 in the light outcoupling surface 8. The recesses 16 are configured to scatter and thus redirect the electromagnetic radiation 3 propagating inside the light guide 6. In particular, the electromagnetic radiation 3 is scattered such that it couples out of the light guide 6, preferably in a direction perpendicular to the light outcoupling surface 8. The recesses 16 have an arbitrary shape and a depth in z-direction that is equal or larger than a wavelength of the electromagnetic radiation 3.

The light outcoupling surface 8 and thus the recesses 16 are arranged on a side of the light guide 6 facing the cooling fins 13. The recesses 16 are distributed across the light outcoupling surface 8 such that the external surface 5 is uniformly irradiated by the electromagnetic radiation 3.

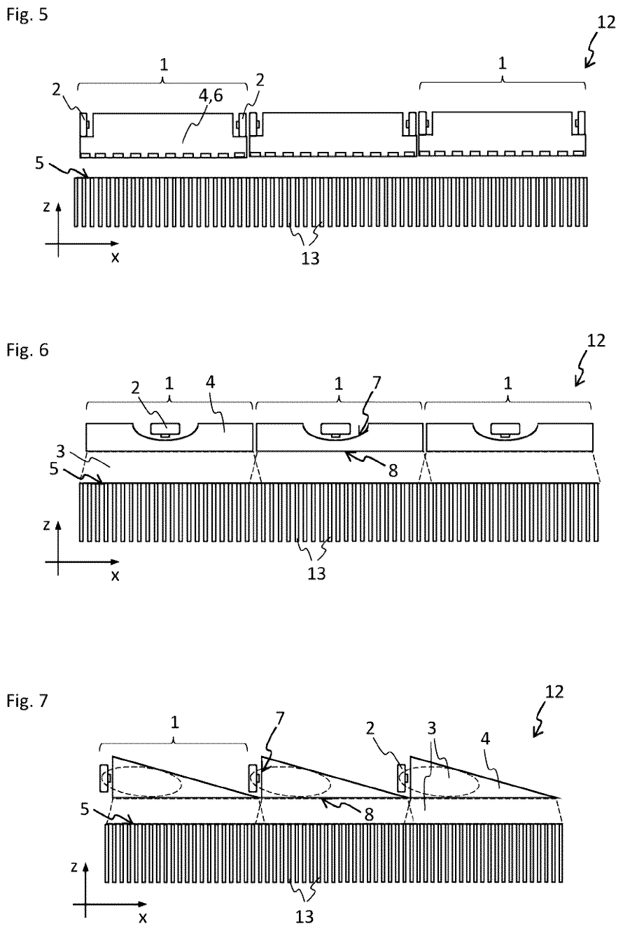

The fluid cooling system 12 according to the exemplary embodiment of FIG. 4 comprises an optoelectronic device 1 with a light guide 6 as described in connection with the exemplary embodiment of FIG. 3. In addition, a reflective element 10 is arranged on a side of the light guide 6 opposite to the structuring 9. Moreover, instead of the second emitter 2 a further reflective element 10 is arranged on a side opposite to the light incoupling surface 7 of the light guide 6. The reflective elements 10 comprise a metallic surface coating of the light guide 6 that is configured to reflect electromagnetic radiation 3 propagating inside the light guide 6 that is incident on the reflective element 10. The reflective elements 10 increase the efficiency of the optoelectronic device 1 by redirecting a larger fraction of the electromagnetic radiation 3 generated by the emitter 2 towards the external surface 5.

The fluid cooling system 12 according to the exemplary embodiment of FIG. 5 comprises a plurality of optoelectronic devices 1 arranged next to each other in x-direction parallel to the external surface 5. In particular, the optoelectronic devices 1 correspond to the optoelectronic device 1 described in connection with the exemplary embodiment of FIG. 3. By arranging a plurality of such optoelectronic devices 1 above the external surface 5, the intensity of the electromagnetic radiation 3 can be increased, or the intensity of the electromagnetic radiation 3 can be kept constant while increasing the area of the external surface 5.

FIG. 6 shows a schematic cross-sectional view in the x-z plane of a fluid cooling system 12 according to a further exemplary embodiment. The fluid cooling system 12 comprises a plurality of cooling fins 13 with main extension planes parallel to the y-z plane, as well as a plurality of optoelectronic devices 1 arranged outside the volume spanned by the cooling fins 13. Electromagnetic radiation 3 emitted by the plurality of optoelectronic devices 1 propagates preferably against the z-direction, parallel to the main extension planes of the cooling fins 13.

The plurality of optoelectronic devices 1 are identical in structure and only one of the optoelectronic devices 1 is described in detail in the following. The optoelectronic device 1 comprises an emitter 2 in the form of a light-emitting semiconductor diode, as well as an optical element 4. The optical element 4 comprises a transparent material for the electromagnetic radiation 3 emitted by the emitter 2 and redirects the electromagnetic radiation 3 towards the external surface 5 due to refraction of the electromagnetic radiation 3. Here, the external surface 5 extends in the x and y directions.

The optical element 4 has a hemispherically, paraboloidically, elliptically or freeform shaped light incoupling surface 7, as well as a planar light outcoupling surface 8 arranged opposite to the light incoupling surface 7. The light outcoupling surface 8 is arranged parallel to the external surface 5 and extends in x and y directions. The light incoupling surface 7 is concave shaped such that a through is formed in the centre of the optical element 4. The emitter 2 is arranged inside the through and emits electromagnetic radiation preferably against the z-direction. In particular, the optical element 4 is configured to redirect electromagnetic radiation 3 emitted by the emitter 2 at large emission angles away from the z-axis towards the external surface 5.

Additionally, a reflective or partially reflective coating can be applied at a region at the centre of the light incoupling surface 7, such that the intensity of the electromagnetic radiation 3 is more uniformly distributed across the light outcoupling surface 8.

The fluid cooling system 12 according to the exemplary embodiment in FIG. 7 comprises a plurality of cooling fins 13 and optoelectronic devices 1 in an arrangement as described in connection with the exemplary embodiment of FIG. 6. Each optoelectronic device 1 comprises an emitter 2 and an optical element 4.

In contrast to the exemplary embodiment descried in connection with FIG. 6, the emitter 2 emits electromagnetic radiation 3 in the x-direction, while the optical element 4 comprises a prism. The prism has a light incoupling surface 7 parallel to the y-z plane and a light outcoupling surface 8 parallel to the x-y plane, i.e. parallel to the external surface 5. The prism redirects the electromagnetic radiation from the light incoupling surface 7 to the light outcoupling surface 8 via total internal reflection at the boundary between the prism and an ambient atmosphere outside the prism, for example. An area of the light outcoupling surface 8 is larger than an area of the light incoupling surface 7 by at least a factor of two.

FIG. 8 shows a schematic cross-section in the x-z plane of a fluid cooling system 12 according to a further exemplary embodiment, similar to the exemplary embodiment described in connection with FIG. 3. In particular, the optical element 4 comprises a cylindrically shaped light guide 6 extending in x-direction as well as two emitters 2 arranged at opposite base surfaces of the cylinder. The base surfaces are parallel to the y-z plane and are configured as light incoupling surfaces 7 of the light guide 6.

In contrast to the exemplary embodiment described in connection with FIG. 3, light outcoupling surface 8 has a structuring 9 along the entire circumference of the light guide 6, such that electromagnetic radiation 3 coupled out of the light guide 6 omnidirectionally, i.e. in all directions. In particular, the electromagnetic radiation 3 is also emitted from the light guide 6 in a direction away from the external surface 5. In order to redirect the electromagnetic radiation 3 towards the external surface 5, the optical element 4 further comprises a reflective element 10 in the form of a plane mirror extending parallel to the x-y plane and thus parallel to the external surface 5. The reflective element 10 is arranged on a side of the light guide 6 opposite to the external surface 5.

FIG. 9 shows a different schematic cross-section along the y-z plane of the fluid cooling system 12 according to the exemplary embodiment of FIG. 8. In particular, a plurality of cylindrical light guides 6 extending in x direction and corresponding emitters 2 are arranged parallel to each other, such that the external surface 5 is uniformly irradiated in y-direction as well.

FIG. 10 shows a schematic cross-section in the x-z plane of a fluid cooling system 12 according to a further exemplary embodiment, similar to the exemplary embodiment described in connection with FIG. 8. In contrast to the exemplary embodiment in FIG. 8, the reflective element 10 is not a plane mirror but instead takes the form of a reflective surface coating on a region of the light outcoupling surface 8 of the cylindrical light guide 6. In particular, the reflective surface coating 10 is applied on one half of the circumference of the cylindrical light guide 6 facing away from the external surface 5. The reflective surface coating comprises Aluminium. Accordingly, the reflective element 10 redirects the electromagnetic radiation 3 towards the external surface 5.

Moreover, FIG. 10 shows a fluid 14, in particular air, flowing between the cooling fins 13. The optoelectronic device 1 is arranged such that the fluid 14 can flow past the optoelectronic device 1 with as little obstruction as possible.

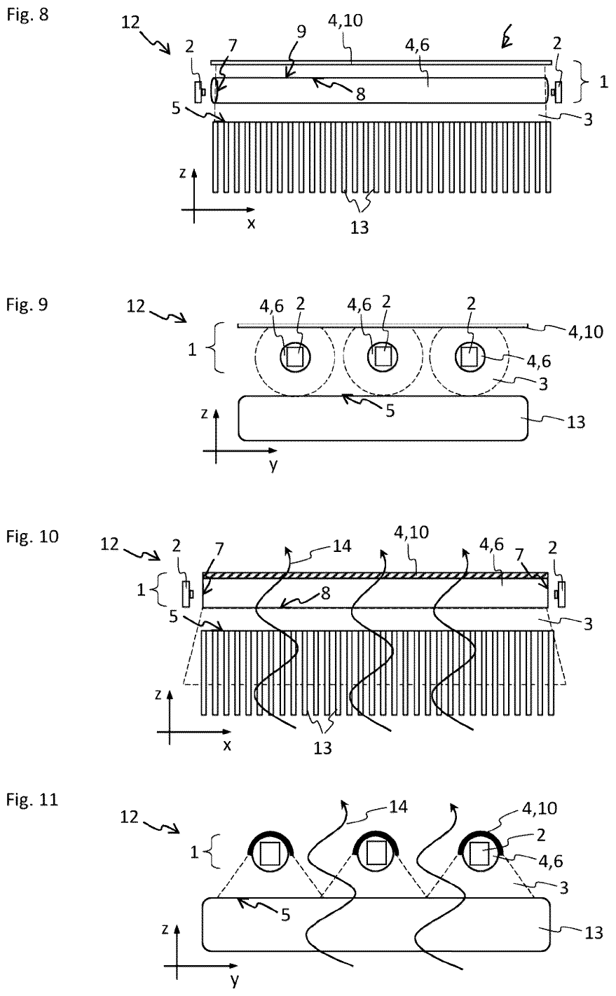

FIG. 11 shows a different schematic cross-section along the y-z plane of the fluid cooling system 12 according to the exemplary embodiment of FIG. 10. In particular, a plurality of cylindrical light guides 6 extending in x direction and corresponding emitters 2 are arranged parallel to each other, such that the external surface 5 is uniformly irradiated in y-direction as well. Moreover, the fluid 14 can flow between the cylindrical light guides 6 through the optoelectronic device 1 without being obstructed.

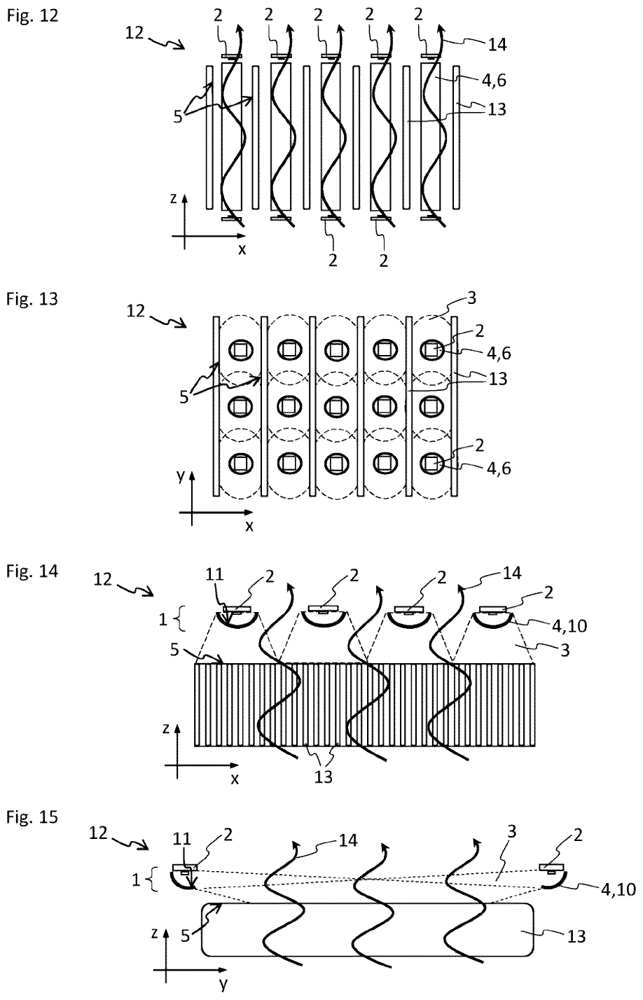

FIG. 12 shows a schematic cross-section in the x-z plane of a fluid cooling system 12 according to a further exemplary embodiment. In contrast to the exemplary embodiment described in connection with FIG. 8 a plurality of cylindrical light guides 6 with corresponding emitters 2 is arranged between each pair of cooling fins 13, such that each light guide 6 extends in z direction. The optoelectronic device 1 is thus arranged inside the volume spanned by the plurality of cooling fins 13 and the electromagnetic radiation 3 is incident directly on the main surface of the cooling fins 13. Accordingly, in this exemplary embodiment no reflective element 10 is needed and the external surface 5 that is uniformly irradiated by the electromagnetic radiation 3 is the main surface of each cooling fin 13 extending parallel to the y-z plane.

FIG. 13 shows a different schematic cross-section along the x-y plane of the fluid cooling system 12 according to the exemplary embodiment of FIG. 12. In particular, a plurality of cylindrical light guides 6 extending in z direction and corresponding emitters 2 are arranged parallel to each other between each pair of cooling fins 13, such that the external surface 5 is uniformly irradiated in y direction as well.

FIG. 14 shows a schematic cross-section in the x-z plane of a fluid cooling system 12 according to a further exemplary embodiment, comprising a plurality of cooling fins 13 with main extension planes extending parallel to the y-z plane. The external surface 5 is a side surface of the volume spanned by the plurality of cooling fins 13 extending parallel to the x-y plane. The optoelectronic device 1 comprises emitters 2 and optical elements 4 in the form of reflective elements 10, such that each emitter 2 has precisely one corresponding reflective element 10. The emitters 2 emit the electromagnetic radiation 3 against the z-direction onto the reflective surfaces 11 of the corresponding reflective elements 10. The reflective element 10 redirects the electromagnetic radiation 3 emitted by the corresponding emitter 2 towards the external surface 5.

The emitters 2 and the reflective elements 10 do not overlap with the cooling fins 13 in plan view of the external surface 5 along the z direction. Moreover, the emitters 2 and the reflective elements 10 do not overlap with the cooling fins 13 in a side view along the x and/or y directions. Advantageously, the flow of the fluid 14 is thus not obstructed by the optoelectronic device 1.

Each reflective element 10 has a size that is comparable to the size of the corresponding emitter 2. In particular, an area of the reflective surface 11 of each reflective element 10 is much smaller than an area of the external surface 5. For example, the area of the external surface 5 is larger than the area of the reflective surface 11 of each reflective element 10 by at least a factor of ten.

The reflective surface 11 of each reflective element 10 is a freeform surface that is neither spherical, paraboloidal nor hyperboloidal. In particular, the shape of the freeform surface is optimized such that the external surface 5 is uniformly irradiated by the electromagnetic radiation 3 emitted by the emitters 2.

FIG. 15 shows a different schematic cross-section along the y-z plane of the fluid cooling system 12 according to the exemplary embodiment of FIG. 14. The reflective surfaces 11 are concave shaped and redistribute the electromagnetic radiation 3 primarily along the y direction of the external surface 5. Preferably, the reflective elements 10 collimate the electromagnetic radiation 3 in x direction such that the electromagnetic radiation 3 can propagate deeper between the cooling fins 13 against the z-direction, thereby disinfecting the fluid 14 flowing between the cooling fins 13.

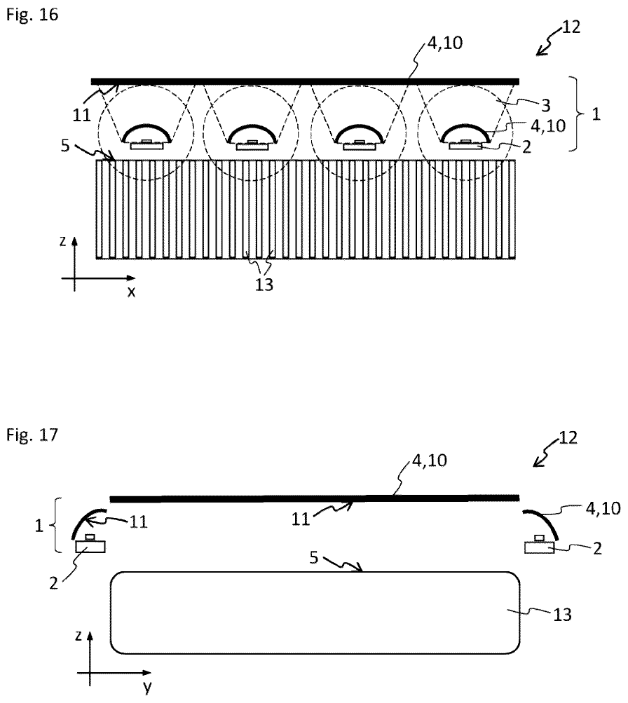

FIG. 16 shows a schematic cross-section in the x-z plane of a fluid cooling system 12 according to a further exemplary embodiment, similar to the exemplary embodiment described in connection with FIGS. 14 and 15. In contrast to the exemplary embodiment in FIG. 14 the emitters 2 emit the electromagnetic radiation 3 in the z-direction onto the reflective surfaces 11 of the corresponding reflective elements 10. The latter have reflective surfaces 11 with a same or a similar shape as the reflective elements 10 described in connection with the exemplary embodiment in FIG. 14.

The reflective elements 10 redirect the electromagnetic radiation 3 onto a further reflective element 10 that is a plane mirror extending parallel to the external surface in the x-y plane. The mirror further redirects the electromagnetic radiation 3 towards the external surface 5. The mirror may be a plane mirror or a mirror with a roughened reflective surface 11 configured for diffuse scattering of the incident electromagnetic radiation 3.

FIG. 17 shows a different schematic cross-section along the y-z plane of the fluid cooling system 12 according to the exemplary embodiment of FIG. 16. Analogous to the embodiment described in connection with FIG. 15, the reflective elements 10 are configured to collimate the electromagnetic radiation 3 in x-direction.

The invention is not restricted to the exemplary embodiments by the description on the basis of said exemplary embodiments. Rather, the invention encompasses any new feature and also any combination of features, which in particular comprises any combination of features in the patent claims and any combination of features in the exemplary embodiments, even if this feature or this combination itself is not explicitly specified in the patent claims or exemplary embodiments.

This patent application claims the priority of German patent application DE 102022118392.0, the disclosure content of which is hereby incorporated by reference.

### REFERENCES

1 optoelectronic device
2 emitter
3 electromagnetic radiation
4 optical element
5 external surface
6 light guide
7 light incoupling surface
8 light outcoupling surface
9 structuring
10 reflective element
11 reflective surface
12 fluid cooling system
13 cooling fin
14 fluid
15 carrier
16 recess
D1 distance between neighboring emitters
D2 distance between emitter and external surface

1. Optoelectronic device, comprising:
at least one emitter, configured for emitting electromagnetic radiation in an ultraviolet spectral range, and
at least one optical element configured for redirecting the electromagnetic radiation onto an external surface, such that the external surface is uniformly irradiated, wherein
the optical element comprises a reflective element with a reflective surface that is a freeform surface.

2. Optoelectronic device according to claim 1,
wherein the emitter is a light emitting semiconductor diode.

3. Optoelectronic device according to claim 1,
comprising at least two emitters, wherein a distance between the two emitters is at least two times larger than a minimal distance between one of the emitters and the external surface.

4. Optoelectronic device according to claim 1, wherein
the optical element comprises a light guide with a light incoupling surface and a light outcoupling surface for the electromagnetic radiation, and
the light outcoupling surface is larger than the light incoupling surface by at least a factor of two.

5. Optoelectronic device according to the previous claim 4,
wherein,
the light outcoupling surface has a structuring such that the electromagnetic radiation is scattered out of the light guide.

6. Optoelectronic device according to claim 5,
wherein,
the structuring is configured to compensate an intensity gradient of the electromagnetic radiation inside the light guide, such that an intensity of the outcoupled electromagnetic radiation is uniform across the light outcoupling surface.

7. Optoelectronic device according to claim 1, wherein
the optical element comprises light scattering particles embedded in a transparent matrix material.

8. (canceled)

9. Optoelectronic device according to claim 1,
wherein
the emitter and the reflective element do not overlap with the external surface in a plan view of the external surface and/or in a side view of the external surface.

10. Optoelectronic device according to claim 1, wherein
an area of the external surface is at least ten times larger than an area of the reflective surface of the reflective element.

11. (canceled)

12. Optoelectronic device according to claim 1, wherein
the external surface has an aspect ratio larger than 2, and
the reflective element collimates the electromagnetic radiation along a short axis of the external surface and homogenizes an intensity profile of the electromagnetic radiation along a long axis of the external surface.

13. Fluid cooling system, comprising:
an optoelectronic device comprising at least one emitter configured for emitting electromagnetic radiation in an ultraviolet spectral range and at least one optical element configured for redirecting the electromagnetic radiation onto an external surface, such that the external surface is uniformly irradiated,
at least two cooling fins arranged parallel to each other, configured to cool a fluid flowing between the two cooling fins, wherein
the electromagnetic radiation emitted by the optoelectronic device irradiates the fluid between the two cooling fins during operation.

14. Fluid cooling system according to claim 11, wherein,
the optoelectronic device is arranged outside a volume delimited by the two cooling fins, and
the external surface irradiated by the electromagnetic radiation during operation corresponds to a cross-sectional surface of the volume through which the fluid flows.

15. Fluid cooling system according to claim 13, wherein
the optoelectronic device is arranged between the two cooling fins such that the external surface irradiated by the electromagnetic radiation during operation corresponds to a main surface of at least one of the cooling fins.
